(19) United States
(12) Patent Application Publication
(10) Pub. No.: US 2022/0024051 A1
(43) Pub. Date: Jan. 27, 2022

(54) A MECHANICAL HAND

(71) Applicant: COVVI Limited, Harrogate (GB)

(72) Inventors: Edward William VARLEY, Leeds (GB); Martin WALLACE, Knaresborough (GB)

(21) Appl. No.: 17/278,965

(22) PCT Filed: Sep. 13, 2019
(86) PCT No.: PCT/GB2019/052571

§ 371 Date: Mar. 23, 2021

(51) Int. Cl. B25J 15/00; B25J 19/00

(52) U.S. Cl. CPC B25J 15/0009; B25J 19/0083; B25J 15/0033

(57) ABSTRACT

The present application describes a cover assembly (102) for a prosthetic or robot hand, comprising a substantially flexible cover portion (104) locatable on at least one moveable digit assembly of a prosthetic hand; and at least one substantially rigid cover element (208, 210, 212, 214) attachable to the flexible cover portion. A prosthetic or robot hand comprising a cover assembly and a method of assembling a prosthetic or robot hand are also described.

The present invention relates to a mechanical hand and in particular, but not exclusively, to a cover assembly for a mechanical hand such as an automated prosthetic or robot hand.

Prosthetic hands, whether they are body-controlled or myo-electrically controlled, typically include a cover located over the electro-mechanical components of the hand. Conventional covers are available in a variety of forms and styles and are typically configured for aesthetic purposes and/or to add structure to the hand and/or to protect the components of the hand from water/dirt ingress, impacts, or the like.

Some conventional covers form a structural part of the hand, whilst hiding the mechanical form underneath and providing the prosthetic hand with a more natural appearance. Due to the structural function, these structural covers are typically made from a relatively stiff material, normally PVC and are around 2-3 mm thick which makes them relatively tough and durable. These covers are retained on the hand via a relatively stiff plastic ring located in an annular groove of the wrist region of the cover which is pulled over a wrist plate on the hand.

Some conventional covers are for purely aesthetic/waterproofing purposes and are available in many different colours to match a person's skin tone. They may also include finger nails and other elements to make them appear more lifelike. These covers are relatively thin (around 1-2 mm) and flexible, and are typically manufactured from silicone. These covers/gloves are also generally very close fitting to the substrate beneath and are retained by friction between the inner surfaces of the glove and the outer surface of the digits and palm region of the prosthetic hand.

Most conventional covers/gloves are manufactured using a slush/rotational moulding process. The slush/rotational moulding process is a manual moulding process where liquid polymer is poured into a female mould, rotated to cover the inside surface of the mould and allowed to cure. This process is repeated numerous times to build up the layers of the glove. The tool is then placed in an oven to cure, once cured the glove is manually pulled from the mould and has a Nylon™ liner bonded in place. Once the Nylon™ liner is cured, the glove can be hand-painted to provide a more realistic appearance.

Gloves that are not slush/rotational moulded are typically injection moulded. This process provides a more consistent thickness and finish, but leaves witness lines where the pieces of the tool meet and is not suitable to form the skin detail generally required for aesthetic gloves.

Furthermore, slush/rotational moulding is a highly manual process and results in expensive and inconsistence covers which can inhibit the performance of the hand. Chemicals such as toluene or xylene are required to create shear in the silicone, allowing it to flow into the moulds. These chemicals are highly toxic and require special care for handling and disposal. Injection moulded gloves are much more consistent but they do have witness marks and the tooling is particularly expensive, approximately twenty-five times the cost of a slush moulded tool.

As prosthetic hands become more advanced with added dexterity, materials such as PVC have to be discounted due to the additional flexibility required by the cover. The relatively thin aesthetics/water barrier covers have a relatively short life, typically less than three months. They frequently move around on the hand, e.g. twisting at the finger tips which adversely affects grip. The cover also migrates forward towards the fingers as the hand is used which can stop the fingers from opening fully and prevent the user from grasping large items, without first having to readjust the cover position.

Aesthetic covers do not closely resemble a human hand and the colour and general aesthetics rarely match a user's other hand. Furthermore, to achieve the flexibility required, aesthetic covers can be particularly baggy around the palm and thumb which again is not desirable.

Migration, bunching up, or the like of the flexible cover particularly at the hinge points of the digits of a hand can result in nipping, cutting or fatigue failure of the flexible cover which in turn undesirably subjects the internal components of the hand to water/dirt ingress, or the like.

It is an aim of certain embodiments of the present invention to provide a cover assembly for a prosthetic or robot hand which adds structure and functionality to the hand whilst allowing movement where required.

It is an aim of certain embodiments of the present invention to provide a cover assembly for a prosthetic or robot hand which prevents undesirable movement, such as migration or twisting, of the cover with respect to the hand substrate on which it is located.

It is an aim of certain embodiments of the present invention to provide a cover assembly for a prosthetic or robot hand which prevents nipping, cutting, fatigue or the like of the flexible cover portion, particularly at hinge points about which the digits extend and flex.

It is an aim of certain embodiments of the present invention to provide a cover assembly for a prosthetic or robot hand which is lightweight yet robust, which protects the electro-mechanical components of the hand against water/dirt ingress and corrosion, and is aesthetically pleasing/customisable, and easily changeable by a clinician or user without the need for return to a dedicated service centre.

According to a first aspect of the present invention there is provided a cover assembly for a prosthetic or robot hand, comprising:
a substantially flexible cover portion locatable on at least one moveable digit assembly of a prosthetic hand; and
at least one substantially rigid cover element attachable to the flexible cover portion.

Optionally, the at least one rigid cover element is removably attachable to the flexible cover portion.

Optionally, at least a portion of the at least one rigid cover element circumferentially extends around a major portion of the flexible cover portion.

Optionally, the flexible cover portion comprises a recessed region for receiving the rigid cover element.

Optionally, the recessed region is correspondingly shaped with the rigid cover element.

Optionally, the rigid cover element comprises at least one projection for engagement with the recessed region of the flexible cover portion.

Optionally, the rigid cover element is configured to be substantially resilient.

Optionally, the flexible cover portion comprises at least one locating region for engagement with a corresponding locating region of the digit assembly.

Optionally, the flexible cover portion is configured to envelope all digit assemblies of the hand and terminate proximal a wrist region of the hand.

Optionally, a wrist region of the flexible cover portion is configured to sealingly couple with the wrist region of the hand.

Optionally, the flexible cover portion comprises at least one bellowed region locatable at an axis of rotation of the digit assembly.

Optionally, the cover assembly further comprises a pulp element locatable on the digit assembly and including a substantially resilient pulp portion.

Optionally, the flexible cover portion comprises a natural or synthetic rubber.

Optionally, the rigid cover element comprises a thermoplastic polymer.

Optionally, the at least one rigid cover element is selectable from a plurality of rigid cover elements.

According to a second aspect of the present invention there is provided a prosthetic or robot hand comprising:
at least one moveable digit assembly;
a substantially flexible cover portion located on the moveable digit assembly; and
at least one substantially rigid cover element attached to the flexible cover portion.

Optionally, the at least one moveable digit assembly comprises at least one phalange member rotatable about an axis and the at least one rigid cover element is attached to the flexible cover portion to at least partially surround the phalange member.

Optionally, the at least one rigid cover element is located in a correspondingly shaped recessed region of the flexible cover portion.

Optionally, the hand further comprises a pulp element located on the phalange member and including a substantially resilient pulp portion.

Optionally, the pulp element comprises a base portion having a first locating region engaged with a correspondingly shaped second locating region of the phalange member.

Optionally, the first locating region comprises a projecting region and the second locating region comprises a recess or aperture.

Optionally, the base portion comprises a recessed region for engagement with a correspondingly shaped projecting region of the flexible cover portion.

Optionally, the flexible cover portion comprises a bellowed region surrounding the axis about which the digit assembly is moveable.

Optionally, the flexible cover portion envelopes all digit assemblies of the hand and terminates proximal a wrist region of the hand.

According to a third aspect of the present invention there is provided a method of assembling a prosthetic or robot hand, comprising:
locating a substantially flexible cover portion on at least one moveable digit assembly of a prosthetic hand; and
attaching at least one substantially rigid cover element to the flexible cover portion.

Optionally, the method comprises locating the at least one rigid cover element in a correspondingly shaped recessed region of the flexible cover portion.

Optionally, the method comprises locating the at least one rigid cover element on the flexible cover portion to at least partially surround a phalange member of the underlying digit assembly.

Optionally, the method further comprises locating a bellowed region of the flexible cover portion around an axis about which the digit assembly is moveable.

Optionally, the method further comprises locating a pulp element on the phalange member of the digit assembly, wherein the pulp element comprises a substantially resilient pulp portion for engagement with the flexible cover portion.

Optionally, the method further comprises engaging an edge region of the rigid cover element with a correspondingly shaped engagement surface of the pulp element.

According to a fourth aspect of the present invention there is provided a prosthetic or robot hand comprising:
at least one moveable digit assembly comprising a phalange member; and
at least one pulp element located on the phalange member, wherein the pulp element comprises a substantially resilient pulp portion.

Optionally, the hand further comprises a force sensor located on the phalange member and engageable with the at least one pulp element.

Optionally, the hand further comprises a substantially flexible cover portion located over the phalange member and the pulp element.

Optionally, the flexible cover portion interlocks with the pulp element.

Optionally, the hand further comprises at least one substantially rigid cover element located on the flexible cover portion to at least partially surround the underlying phalange member.

Optionally, the rigid cover element comprises an aperture to expose the flexible cover portion engaging the pulp portion of the pulp element.

### DESCRIPTION OF THE DRAWINGS

Certain embodiments of the present invention will now be described with reference to the accompanying drawings in which:

FIG. 1a illustrates the back of a prosthetic hand comprising a cover assembly according to certain embodiments of the present invention;

FIG. 1b illustrates the front of the prosthetic hand of FIG. 1a;

FIG. 2 illustrates an exploded view of the cover assembly;

FIG. 3a illustrates a finger portion of the cover assembly;

FIG. 3b illustrates the underside of a fingertip pulp element of the finger portion of the cover assembly of FIG. 3a for engagement with the distal phalange member of the finger assembly;

FIG. 3c illustrates the fingertip pulp element engaged with the distal phalange member of the finger assembly;

FIG. 3d illustrates a flexible cover portion located over the sub-structure including the finger assembly and fingertip pulp element;

FIG. 3e illustrates a rigid cover element for locating on the flexible cover portion; and

FIG. 3f illustrates a section through the distal phalange cover assembly.

### DETAILED DESCRIPTION

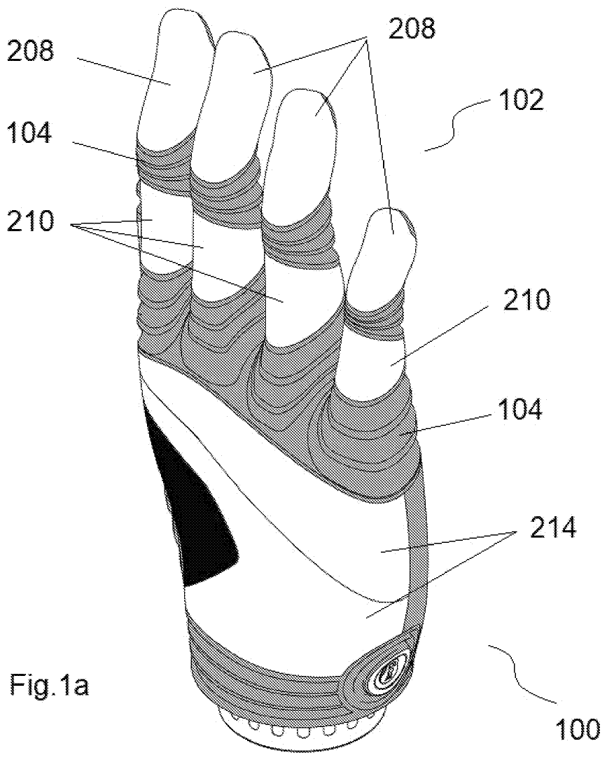

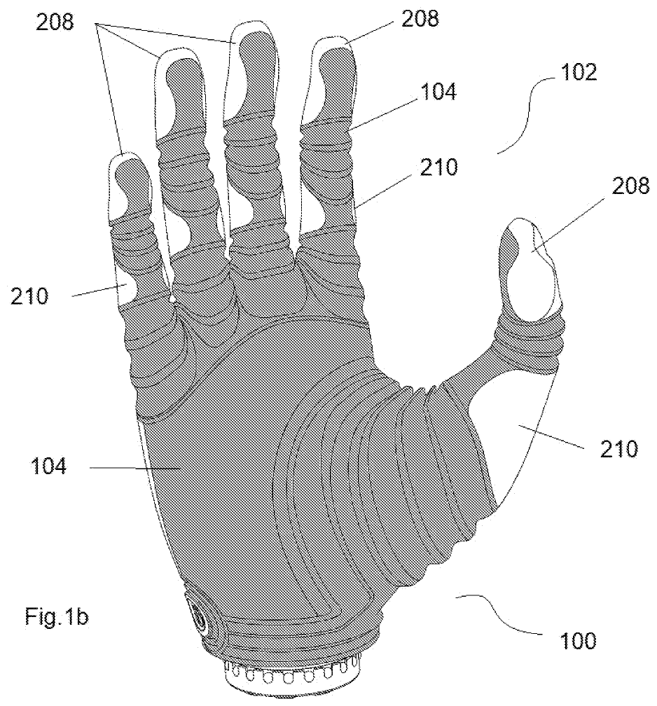

As illustrated in FIGS. 1a and 1b, a prosthetic hand 100 according to certain embodiments of the present invention includes a cover assembly 102 having a substantially flexible cover portion 104 located over the chassis/working components of the hand, and a plurality of substantially resilient and relatively rigid cover elements 208,210,212,214 mounted on the flexible cover portion 104. The rigid cover elements 208,210,212,214 are located over regions of the hand 100 which are relatively rigid, e.g. the phalange regions of each digit and the palm and dorsal regions of the hand, and adjacent to or between other regions of the hand about which the rigid regions are rotated, e.g. the proximal interphalangeal joint and the metacarpophalangeal joint of each digit assembly, the carpometacarpal joint of the thumb assembly, and the wrist joint.

The chassis and working components of the prosthetic hand are fully contained within the flexible hand-shaped cover portion 104. Aptly the flexible cover portion is formed of a substantially flexible yet resilient material, such as neoprene, and optionally comprises impregnated graphene to provide additional flexibility and strength. Alternatively, the flexible cover portion may be manufactured from another suitable material, such as latex or a bio-compatible variant of latex, for example. The flexible cover portion is aptly dip-moulded but may be manufactured by another suitable process such as slush, rotational or injection moulding, for example. The flexible cover portion is around 0.5-1 mm thick and is a one-piece closed unit to fully cover and protect the chassis and electromechanical components from water/dirt ingress.

The elasticity of the flexible cover/glove portion 104 allows it to be easily applied over the underlying structure of the hand during manufacturer and also applied and removed by the user. The elasticity of the flexible cover portion 104 allows each portion of the finger and thumb assemblies to move unimpeded about their respective axes. The flexible cover portion 104 follows the underlying structure of the hand closely. For example, where the structure is relatively rigid, e.g. the metacarpal region or the individual phalanges, the form of the flexible cover portion closely engages with the underlying structure of the hand, but at the regions where the hand mechanism flexes, i.e. around the digit axes, the flexible cover portion includes bellowed regions 106 comprising ridges and folds that allow the material of the flexible cover portion to move with respect to the sub-structure of the hand, whilst still allowing for the overall form of the hand to be maintained in all positions and grip patterns of the hand in use. Aptly, the inner surface of the flexible cover portion 104 may include one or more protrusions for location in respective recesses/openings in the sub-structure of the hand to thereby securely locate the flexible cover portion with respect to the underlying sub-structure and prevent migration of the same during use. Dip moulding is a suitable method to form such elements.

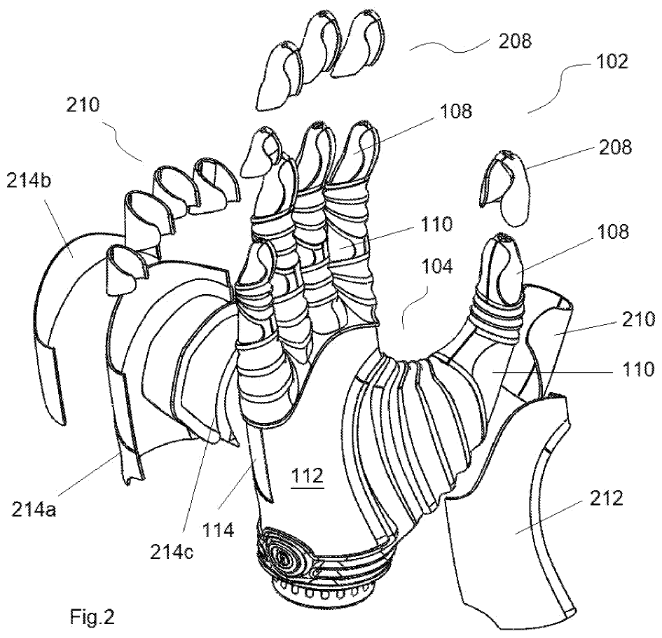

As illustrated in FIG. 2, the flexible cover portion 104 includes a plurality of recessed regions 108,110,112,114 each configured to receive a respective and correspondingly shaped one of the substantially resilient and relatively inflexible/stiff cover elements 108,110,112,114. For example, as illustrated in FIG. 2, each distal phalange portion of the flexible cover portion 104 includes a recessed region 108 to receive a correspondingly shaped rigid cover element 208. Likewise, each middle phalange portion of the flexible cover portion 104 includes a recessed region 110 to receive a correspondingly shaped rigid cover element 210. Furthermore, the ventral region (palm) 112 and the dorsal region (back) 114 of the flexible cover portion 104 are also recessed to receive correspondingly shaped rigid ventral and dorsal cover elements 212,214 respectively.

The dorsal cover elements 214 comprise a primary cover element 214a and a secondary cover element 214b which locates on the outer surface of the primary cover element by bonding or the like. The secondary cover element may be purely aesthetic and removably attached to the primary cover element to be customisable/interchangeable. One of the dorsal cover elements 214c is aptly a display, such as a touch screen, to allow a user to operate the hand via the same either alone or in conjunction with conventional myo-sensors located in the limb socket. For example, a user may select a desired grip pattern displayed on the touch screen by using a finger of an able hand and/or the display may provide an indication to the user which grip pattern has been selected and/or which is selectable and/or the display may aid the user in selecting a desired grip pattern such as by displaying a list of selectable grip patterns. The display may also be configured to selectively display a desired colour/graphic/image to further customise the appearance of the cover.

The palm or ventral cover element 212 is aptly a two-part moulding comprising a relatively hard plastic substrate layer, such as Nylon™ or Acrylonitrile Butadiene Styrene (ABS), to provide form and structure, and an over-moulded flexible polymer layer to provide improved grip which may comprise a thermoplastic elastomer, rubber, or the like.

The rigid cover elements 208,210,212,214 are one-piece injection moulded plastic elements, such as Nylon™ or ABS, or the like. Alternatively, each rigid element may be made by machining, casting, pressing, metal injection moulding, 3D printing, or the like, and may be another suitable metal or plastics material, such as aluminium, stainless steel, HDPE, polypropylene, or the like. The rigid cover elements may also be any desired texture and/or colour to provide the hand with a desired and customisable appearance and/or function. For example, the cover elements may be luminous for low light applications, such as walking or cycling at night. They may be painted, screen printed, pad printed, hydrographically printed, or the like.

The rigid cover elements 208,210,212,214 are aptly clipped and optionally bonded in place on the flexible cover portion104. They are configured to snap-fit at least partially around the flexible cover portion to securely be located thereon.

Alternatively, they may be bolted, screwed, pinned, or secured in place by magnets to allow them to be removably attached to the flexible cover portion for ease of interchangeability/customisation if desired.

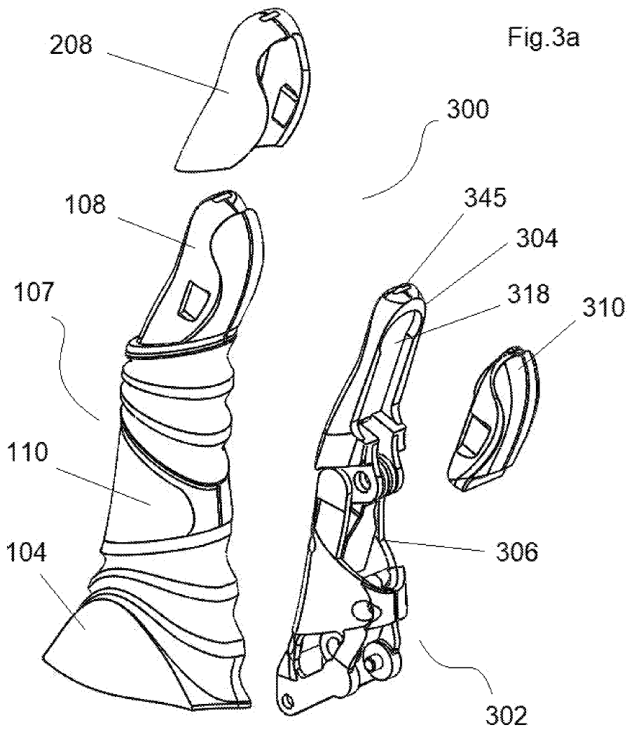

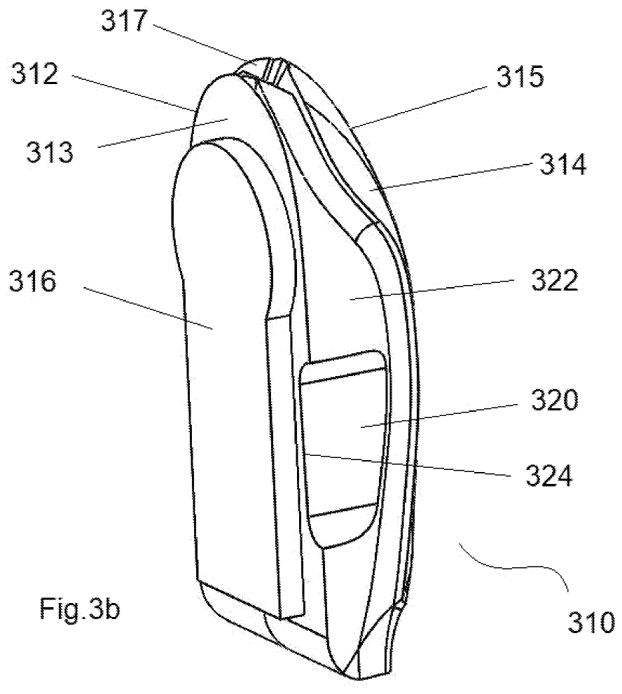

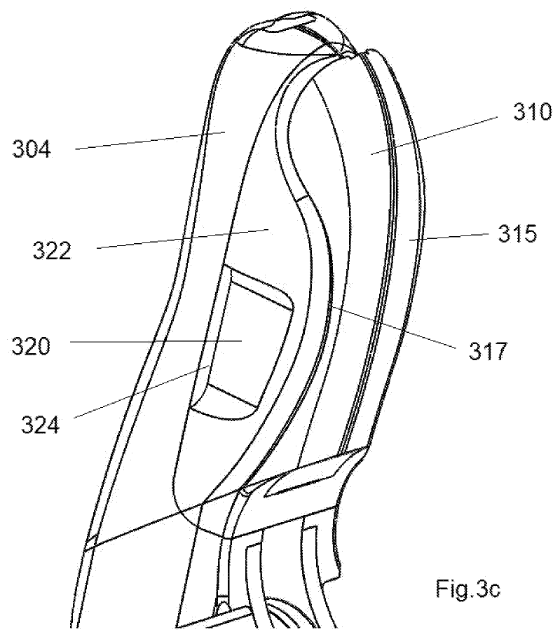

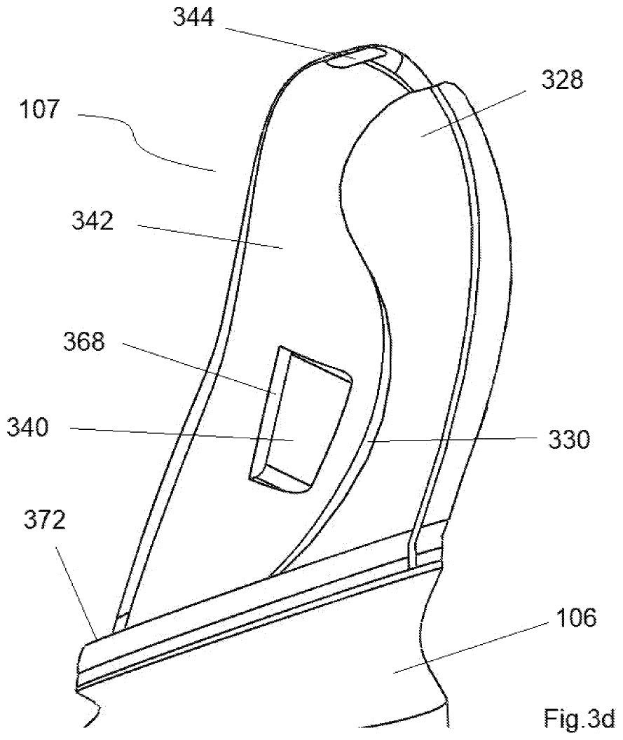

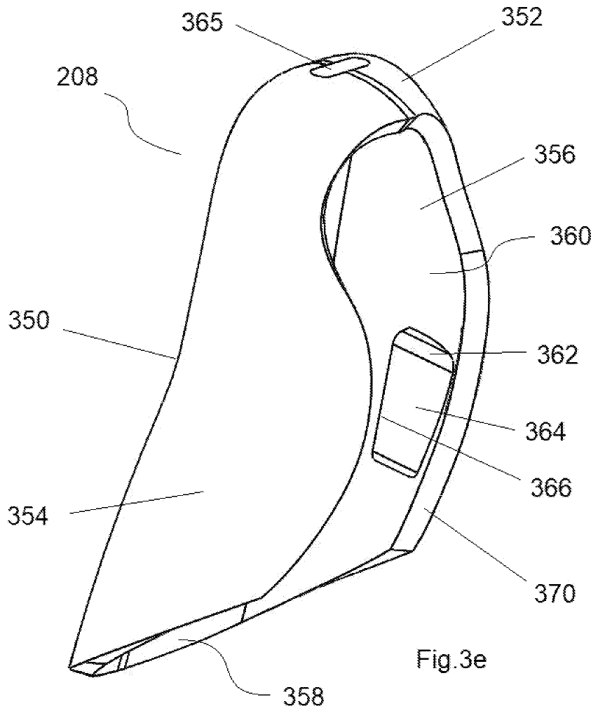

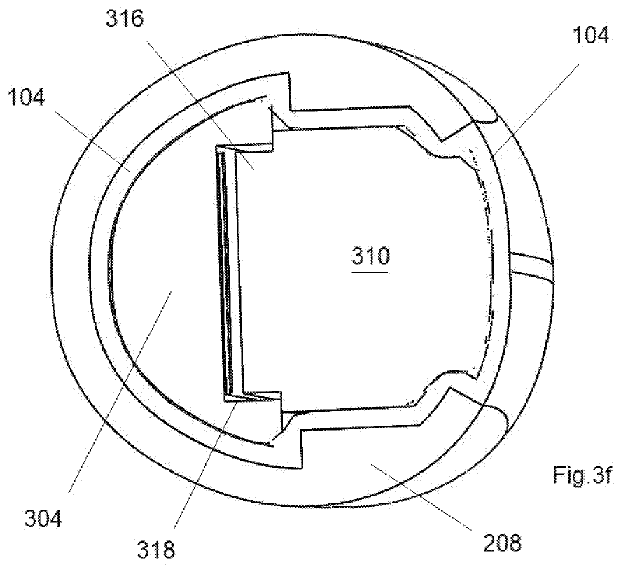

As an example, an index finger assembly 300 according to certain embodiments of the present invention is illustrated in FIG. 3a in exploded form. The finger assembly 300 includes a mechanical arrangement 302 comprising a plurality of pivotally coupled linkage members 304,306 which respectively resemble the distal and proximal phalanges of a natural hand. The assembly further includes a resilient pulp element 310 mountable on the distal phalange member 304, a phalange portion 107 of the flexible cover portion 104 locatable over the finger substructure including the mechanical arrangement 302 and the pulp element 310, and a rigid cover element 208 locatable over the flexible cover portion 104.

As illustrated in FIG. 3b, the pulp element 310 includes a base portion 312 and a pulp portion 314. At least the pulp portion 314 is configured to be substantially resilient in that it is spongy to resemble the pulp region of a natural fingertip and provide improved grip and touch functionality to the prosthetic hand in use. The pulp element 310 is aptly an integral one-piece element formed of a plastics material, such as flexible TPE or silicone or the like. Alternatively, the base portion 312 may be a relatively rigid material, such as Nylon™ or ABS, and the pulp portion 314 may comprise a more spongy and resilient material. The pulp portion 314 may be made up of a plurality of different components, such as a resilient substrate and a cover layer.

The edge of the pulp portion 314 extends beyond the sides of the base portion 312 to define a lip 317 extending around the two sides and distal end region of the pulp element 310. The base portion 312 of the pulp element 310 further includes a keyhole-shaped projecting region 316 outwardly extending from a base surface 313 thereof for engagement with a correspondingly shaped aperture 318 of the distal phalange member 304 (see FIG. 3a). The corresponding shape of the projecting region 316 and the aperture 318 securely locate the pulp element 310 on the distal phalange member 304 in a single orientation to ensure the same is always oriented correctly and to prevent the same rotating with respect to the distal phalange member 304 when mounted thereon. Other suitable shapes and/or sizes of projecting region 316 and aperture 318 to provide such an arrangement can be envisaged, such as orthogonal, oval, or the like. The pulp element 310 is aptly adhered to the distal phalange member 304 or otherwise secured thereto such as by mechanically fastening, a snap-fit connection, or the like.

The projecting region 316 of the pulp element 310 is also configured to engage in use with a force sensitive resistor (FSR) (not shown) located in the aperture of the distal phalange member 304. The resilience of the pulp element 310, particularly when a one-piece element of resilient material, allows a force applied to the outer surface of the flexible cover portion 104 to be transferred through the pulp element 310 to the FSR. Alternatively, or additionally, the pulp element 310 may be translatable with respect to the FSR when mounted to the distal phalange member 304 to thereby engage the FSR or similar force/load sensor when a force is applied thereto. Further alternatively, a pulp element may not be present and the FSR or similar may engage directly with an inner surface of the flexible cover portion, or the flexible cover portion itself may comprise a pulp portion for engagement with the FSR.

The pulp element 310 further includes a locating region in the form of a recessed region 320 on each lateral side surface 322 with respect to a longitudinal axis of the grip element. Each recessed region 320 defines a shoulder portion providing an engagement surface 324 proximal the base surface 313 of the pulp element 310.

FIG. 3c illustrates the pulp element 310 mounted on the distal phalange member 304.

As illustrated in FIG. 3d, the flexible cover portion 104 is located over the substructure 302 and the pulp element 310. The flexible cover portion 104 includes the bellowed region 106 to allow the finger members 304,306 to rotate relative to each other in use, and also a fingertip region 107 which is sized and shaped to closely engage with the underlying outer surfaces of the distal phalange member 304 and the pulp element 310. The fingertip region 107 of the flexible cover portion 104 includes a stepped outer surface 328 to define a lip/overhang and in turn a curved abutment surface 330 for close engagement with the corresponding lip 317 of the pulp element 310. The stepped outer surface 328 may be the same or a different material to the main body of the flexible cover portion 104, e.g. the stepped outer surface may be a material, such as rubber, having a relatively high coefficient of friction with respect to other regions of the flexible cover portion, such as the bellowed regions 106, to thereby provide additional grip at the tip regions of each digit.

The fingertip region 107 also includes a locating region in the form of a recessed region 340 on each lateral side surface 342 thereof with respect to a longitudinal axis of the finger assembly which each define a projecting region (not shown) on respective inner surfaces of the flexible cover portion 104 for respective engagement with the recessed regions 320 of the pulp element 310. The flexible cover portion 104 also optionally includes a recessed region 344 at the fingertip end region which defines a projecting rib (not shown) on the inner surface of the flexible cover portion for engagement with a corresponding recess 345 of the distal phalange member 304 (see FIG. 3a).

The recessed regions 320,340 and corresponding projections are substantially rectangular but may be any suitable configuration, such as circular, ribbed, oval, tooth-like, or the like, to provide security when the flexible cover portion is located on the substructure and when the rigid cover element 208 is located on the flexible cover portion 104, as described further below. Each recessed region 320,340 may be a single recessed region as illustrated or comprise a plurality of recessed regions on each side of the pulp element 310 and flexible cover portion 104 respectively. This arrangement of recesses and projections securely locates the flexible cover portion 104 on the substructure 302 to prevent movement of the flexible cover portion 104 with respect to the substructure in use. Additional locating regions, such as further projections or recesses, may be provided on the inner surface of the flexible cover portion 104 for engaging with corresponding recesses/apertures or projections provided elsewhere on the substructure of the hand, such as the palm/dorsal regions.

As illustrated in FIG. 3e, a rigid cover element 208 according to certain embodiments of the present invention for locating on the distal phalange region of the flexible cover portion 104 includes a back portion 350, a first end portion 352, a pair of opposed side portions 354,356, a substantially open second end portion 358 disposed opposite the first end portion, and a substantially open front portion 360 disposed opposite the back portion 350. The inner surfaces of the cover element 208 substantially correspond to the outer surfaces of the flexible cover portion to thereby engage closely therewith when the cover element 208 is located on the flexible cover portion 104. Each inner surface of the side portions 354,356 includes a tapered projecting region 362 defining a tapered engagement surface 364 and a latching surface 366. Each projecting region 362 is sized and shaped to correspondingly engage with the recessed regions 340 on each side of the flexible cover portion 104 (see FIG. 3d) such that when the rigid cover element 208 is pushed onto the fingertip region 107 of the flexible cover portion 104, each tapered engagement surface 364 slides over a respective one of the outer side surfaces 342 of the flexible cover portion 104 until aligned with the recessed regions 340 therein at which point the resilience of the cover element 208 urges the tapered projections 362 into the corresponding recessed regions 340. The arrangement of the latching surface 366 of each tapered projection 362 and the shoulder 368 defined by each recessed region 340 securely locates the rigid cover element 208 on the flexible cover portion 104 in a correct position and orientation. A further locating element 365 is optionally provided at the first end portion 352 for engagement with the optional recess 344 in the end of the flexible cover portion 104.

When the rigid cover element 208 is located on the flexible cover portion 104, the curved edge region 370 surrounding the opening in the front portion 358 of the rigid cover element 208 engages with the correspondingly curved abutment surface 330 of the flexible cover portion 104. Likewise, the edge of second end portion 358 of the rigid cover element 208 engages with a shoulder region 372 provided adjacent to the bellowed region 106 of the flexible cover portion 104. A substantially flush outer surface of the cover assembly is thereby provided at the interface between the rigid cover element 208 and the flexible cover portion 104.

A cross section through the distal phalange portion of the finger assembly 300 is illustrated in FIG. 3f. The projecting region 316 of the pulp portion 310 engages in the correspondingly shaped aperture 318 of the distal phalange member 304 and is secured thereto by adhesive, mechanical fastening, or the like. The flexible cover portion 104 is located over the substructure 304,310 to conform closely if not exactly therewith. The rigid cover element 208 is then located over the flexible cover portion 104 to sandwich the same between the rigid cover element 208 and the substructure 304,310. The rigid cover element 208 is configured to clip over the flexible cover portion 104 and securely retain itself thereon. For example, a portion of the rigid cover element 208 locates around a major portion, i.e. more than half, of the circumference of the flexible cover portion 104 and the resilience of the rigid cover element 208 allows it to flex during fitment on the flexible cover portion whilst retaining its shape when fully located on the flexible cover portion 104. Such resilience desirably provides a snap-fit connection.

As illustrated in FIG. 3f, the cover assembly provides a sealed barrier to prevent the ingress of water or dirt through the cover assembly and into the electromechanical working components of the prosthetic hand. The outer surface provided by the assembled cover components is substantially flush and continuous to provide an aesthetically pleasing and technically functional cover for the prosthetic hand.

In order to apply the flexible cover portion 104 to the prosthetic hand it will require at least one open end such that the prosthetic hand can be inserted therein. This most naturally would occur at the wrist but could be located elsewhere on the glove, such as on the dorsal side of the glove. An aperture at the wrist region of the flexible cover portion 104 would also allow a securing element or rigid substrate portion of the hand to protrude from the cover assembly for engagement with a corresponding attachment element.

In order to seal the edge of the flexible cover portion 104 against the wrist section, rigid cover elements, such as the palm and dorsal cover elements 212,214, may each comprise one or more lipped edge regions to compress against the flexible cover portion and sealingly clamp the same to the rigid substrate. Preferably, the wrist region of the flexible cover portion 104 terminates at an annular base region having a central aperture therein to define a substantially flat inner engagement surface around the central aperture at the wrist region of the flexible cover portion. A securing element comprising a flanged disc region and an externally threaded boss/shaft is configured to locate through the central aperture such that the flanged disc region engages with the inner engagement of the flexible cover portion. An internally threaded ring or nut element engages on the threaded boss/shaft of the securing element to sealingly clamp the flexible cover portion therebetween. A disc washer may optionally be used to act as a barrier to the rotation of the ring/nut to prevent the flexible cover portion bunching up during fitting. The external ring/nut element may also be configured to suit various methods of protecting the complete prosthetic limb, not just the hand, from water and dust ingress.

The finger cover assembly 300 has been illustrated and described as an example only and the same arrangement can be applied to the other digits, such as the thumb assembly as shown in FIG. 2. Aptly, all the digits of the prosthetic hand include such rigid cover elements located on a flexible cover portion to thereby seal the hand from water/dirt ingress and also secure the flexible cover portion with respect to the underlying substructure at predetermined locations of the hand to prevent migration, rubbing, bunching, fatigue or the like of the flexible cover at one or more of those locations.

The combination of hard/rigid cover elements and a soft/flexible cover portion provides an aesthetically pleasing, flexible and robust cover, whilst also providing an effective water barrier. The one-piece glove is the primary barrier, but is also fixed to a close-fitting substrate preventing water migration. If it is required for the flexible cover portion to include a hole, for example for a screen or switch, the edge of the hole may aptly comprise a vertical rim which would be compressed by the rigid cover element to thereby seal the joint and create an effective water barrier.

Certain embodiments of the present invention therefore provide a cover assembly for a prosthetic or robot hand which adds structure and functionality to the hand whilst allowing movement where required but which prevents undesirable movement, such

as migration, bunching or twisting, of the cover with respect to the hand substrate on which it is located. The cover assembly prevents nipping, cutting, fatigue or the like of the flexible cover portion, particularly at hinge points about which the digits extend and flex. The cover assembly is lightweight yet robust, protects the electro-mechanical components of the hand against water/dirt ingress and corrosion, and is aesthetically pleasing/customisable.

1-36. (canceled)

37. A cover assembly for a mechanical hand, comprising:
a substantially flexible glove-like cover portion locatable on a mechanical hand comprising a plurality of digit assemblies including at least one moveable digit assembly having at least one phalange member rotatable about an axis; and
at least one substantially rigid cover element attachable to the flexible cover portion to at least partially surround the phalange member,
wherein:
the flexible cover portion comprises at least one locating region for engagement with a corresponding locating region of the phalange member to securely locate the flexible cover portion with respect to the phalange member, and
the flexible cover portion comprises at least one recessed region for receiving a corresponding region of the rigid cover element to securely locate the rigid cover element on the flexible cover portion.

38. The cover assembly according to claim 37, wherein the at least one rigid cover element is removably attachable to the flexible cover portion.

39. The cover assembly according to claim 37, wherein at least a portion of the at least one rigid cover element circumferentially extends around a major portion of the flexible cover portion.

40. The cover assembly according to claim 37, wherein the recessed region is correspondingly shaped with the corresponding region of the rigid cover element.

41. The cover assembly according to claim 37, wherein the rigid cover element comprises at least one projection for engagement with the recessed region of the flexible cover portion.

42. The cover assembly according to claim 37, wherein the rigid cover element is configured to be substantially resilient.

43. The cover assembly according to claim 37, wherein the flexible cover portion is configured to envelope all digit assemblies of the hand and terminate proximal a wrist region of the hand.

44. The cover assembly according to claim 43, wherein a wrist region of the flexible cover portion is configured to sealingly couple with the wrist region of the hand.

45. The cover assembly according to claim 37, wherein the flexible cover portion comprises at least one bellowed region locatable at the axis of rotation of the phalange member of the moveable digit assembly.

46. The cover assembly according to claim 37, wherein the flexible cover portion comprises a natural or synthetic rubber, and the rigid cover element comprises a thermoplastic polymer.

47. A mechanical hand comprising:
a plurality of digit assemblies including at least one moveable digit assembly having at least one phalange member rotatable about an axis; and
the cover assembly according to claim 37 located on the hand.

48. The hand according to claim 47, further comprising a pulp element located on the phalange member and including a substantially resilient pulp portion.

49. The hand according to claim 48, wherein the pulp element comprises a base portion having a first locating region engaged with a correspondingly shaped second locating region of the phalange member.

50. The hand according to claim 49, wherein the first locating region comprises a projecting region and the second locating region comprises a recess or aperture.

51. The hand according to claim 49, wherein the base portion comprises a recessed region for engagement with a correspondingly shaped projecting region of the flexible cover portion.

52. A method of assembling a mechanical hand, comprising:
locating a substantially flexible glove-like cover portion on a mechanical hand comprising a plurality of digit assemblies including at least one moveable digit assembly having at least one phalange member rotatable about an axis; and
attaching at least one substantially rigid cover element to the flexible cover portion to at least partially surround the phalange member,
wherein:
locating comprises engaging at least one locating region of the flexible cover portion with a corresponding locating region of the phalange member to securely locate the flexible cover portion with respect to the phalange member, and
attaching comprises receiving in at least one recessed region of the flexible cover portion a corresponding region of the rigid cover element to securely locate the rigid cover element on the flexible cover portion.

53. The method according to claim 52, wherein attaching comprises engaging at least one projection of the at least one rigid cover element in the at least one recessed region of the flexible cover portion.

54. The method according to claim 52, wherein attaching comprises locating the at least one rigid cover element on the flexible cover portion to at least partially surround the phalange member of the underlying moveable digit assembly.

55. The method according to claim 54, comprising locating a bellowed region of the flexible cover portion around the axis about which the digit assembly is moveable.

56. The method according to claim 54, comprising locating a pulp element on the phalange member of the digit assembly, wherein the pulp element comprises a substantially resilient pulp portion for engagement with the flexible cover portion.
